(19) United States
(12) Patent Application Publication
(10) Pub. No.: US 2018/0098932 A1
(43) Pub. Date: Apr. 12, 2018

(54) MIXTURE OF SUBSTANCES FOR STYLING AGENT

(71) Applicant: Henkel AG & Co. KGaA, Duesseldorf (DE)

(72) Inventors: Marcus Noll, Norderstedt (DE); Thorsten Knappe, Schenefeld (DE); Anna Puls, Winsen (Luhe) (DE); Marie Meisel, Hemslingen (DE)

(73) Assignee: Henkel AG & Co. KGaA, Duesseldorf (DE)

(21) Appl. No.: 15/671,145

(22) Filed: Aug. 8, 2017

(30) Foreign Application Priority Data
Oct. 12, 2016 (DE) 10 2016 219 846.7

(51) Int. Cl. A61K 8/88; A61Q 5/06; A61K 8/04; A61K 8/31

(52) U.S. Cl. CPC A61K 8/88; A61Q 5/06; A61K 2800/594; A61K 8/31; A61K 8/046

(57) ABSTRACT

A cosmetic agent for the temporary styling of keratinic fibers, in particular human hair, containing, in a cosmetic support, with respect to its total weight,
a) from about 0.05% to about 5.0% by weight of at least one (meth)acrylamidopropyltrimethylammonium salt homo- or copolymer;
b) from about 0.5% to about 10% by weight of polyimide,
the uses thereof and a method employing this agent.

### CROSS-REFERENCE TO RELATED APPLICATION

This application claims priority to German Patent Application No. 10 2016 219 846.7, filed Oct. 12, 2016, which is incorporated herein by reference in its entirety.

### TECHNICAL FIELD

The present disclosure relates to cosmetic agents for the temporary styling of keratinic fibers which contain, in a cosmetic support, a combination of at least one (meth)acrylamidopropyltrimethylammonium salt homo- or copolymer and at least one polyimide.

### BACKGROUND

The present disclosure further relates to methods for the temporary styling of keratinic fibers as well as the use of the cosmetic agent for improving manageability, the hold of a hairstyle and to straighten the hair.

Agents for the temporary shaping of human hair are known per se. These agents serve quite different cosmetic purposes, from waving or crimping straight hair to straightening frizzy hair.

Irrespectively of the waviness of the hair, customers in all consumer groups are united in their desire for smooth, glossy hair which sits harmoniously with the desired hairstyle, without individual hairs escaping from the hairstyle. Cosmetic methods for obtaining frizz-free hair like this include, for example, the daily use of straighteners and/or setting lotions. The cosmetic effects on the hair which are obtained are usually of short duration.

Longer-lasting frizz-free shaped hairstyles are obtained by using highly alkaline relaxers, formaldehyde or low concentrations of thioglycolate formulations. Broadly, the action of these cosmetics resides in acting on the chemical structure of the hair. Using them can entail unwanted damage to the hair or irritation to the skin of the scalp.

### BRIEF SUMMARY

Cosmetic agents and methods of using the same are provided. In an exemplary embodiment, the cosmetic agent for the temporary styling of keratinic fibers includes, in a cosmetic support, with respect to its total weight: (a) from about 0.05% to about 5% by weight of at least one (meth)acrylamidopropyltrimethylammonium salt homo- or copolymer; and (b) from about 0.5% to about 10% by weight of a polyimide.

A method of temporarily shaping keratinic fibers is provided in another embodiment. The method includes applying a cosmetic agent to the keratinic fibers, where the cosmetic agent includes, in a cosmetic support, with respect to its total weight: (a) from about 0.05% to about 5% by weight of at least one (meth)acrylamidopropyltrimethylammonium salt homo- or copolymer; and (b) from about 0.5% to about 10% by weight of a polyimide.

### DETAILED DESCRIPTION

The following detailed description is merely exemplary in nature and is not intended to limit the disclosure or the application and uses of the subject matter as described herein. Furthermore, there is no intention to be bound by any theory presented in the preceding background or the following detailed description.

Thus, the objective of the present disclosure is to provide care agents for the gentle temporary shaping of keratinic fibers which provide a substantial improvement to the structure and hold of a hairstyle without having to resort to thermal appliances such as straighteners or to chemical aids such as relaxers, formaldehyde or thioglycolate.

The cosmetic effect obtained by employing the agents as contemplated herein should be discernible for several days or even after washing the hair; in other words, the hairstyles obtained should remain frizz-free for a longer period than with conventional hairstyles.

The hairstyles obtained should also exhibit improved combability and an improved feel.

The term “combability” as used in the context of the present disclosure should be understood to mean both the combability of the wet fibers and the combability of the dry fibers. A measure of combability is the work required for combing or the force used during the procedure of combing a bunch of fibers. The measurement parameters can be assessed sensorially by the person skilled in the art or be quantified using measuring devices.

The term “feel” is defined as the tactility of a bunch of fibers, wherein the person skilled in the art feels the parameters of fullness and smoothness of the bunch and assesses it sensorially.

These objectives are achieved by employing a special combination of substances comprising a (meth)acrylamidopropyltrimethylammonium salt homo- or copolymer and a polyimide. Hair care agents based on (meth)acrylamidopropyltrimethylammonium salt homopolymers have, for example, been described in the German patent application DE 10 2014 221 400 A1. Polyimide-containing fixing mousses have been disclosed in the US patent application US 2013/0309190 A1.

The agents and methods as contemplated herein and some of their preferred embodiments are defined in the following points:
1. A cosmetic agent for the temporary styling of keratinic fibers, in particular human hair, containing, in a cosmetic support, with respect to its total weight,
a) from about 0.05% to about 5.0% by weight of at least one (meth)acrylamidopropyltrimethylammonium salt homo- or copolymer;
b) from about 0.5% to about 10% by weight of polyimide.
2. A cosmetic agent according to point 1, exemplified in that it contains a (meth)acrylamidopropyltrimethylammonium salt homopolymer as the (meth)acrylamidopropyltrimethylammonium salt homo- or copolymer a), preferably (3-acrylamidopropyl)-trimethylammonium salt homopolymer, more preferably (3-acrylamidopropyl)-trimethylammonium chloride homopolymer.
3. A cosmetic agent according to point 1, exemplified in that it contains a (meth)acrylamidopropyltrimethylammonium salt homopolymer as the (meth)acrylamidopropyltrimethylammonium salt homo- or copolymer a), preferably (3-methacrylamidopropyl-trimethylammonium salt homopolymer, more preferably (3-methacrylamidopropyl)-trimethylammonium chloride homopolymer.
4. A cosmetic agent according to point 1, exemplified in that it contains a (meth)acrylamidopropyltrimethylammonium salt copolymer as the (meth)acrylamidopropyltrimethylammonium salt homo- or copolymer a), preferably (3-acrylamidopropyl)-trimethylammonium salt copolymer, more preferably (3-acrylamidopropyl)-trimethylammonium chloride-acrylamide copolymer.
5. A cosmetic agent according to one of the preceding points, exemplified in that the proportion by weight of the (meth)acrylamidopropyltrimethylammonium salt homo- or copolymer a), preferably of the (meth)acrylamidopropyltrimethylammonium salt homopolymer a), with respect to the total weight of the cosmetic agent is from about 0.05% to about 4.0% by weight, preferably from about 0.1% to about 3.0% by weight and in particular from about 0.1% to about 2.0% by weight.
6. A cosmetic agent according to one of the preceding points, exemplified in that the polyimide contains at least one structural unit (A1), at least one structural unit (A2), at least one structural unit (A3) and at least one structural unit (A4),

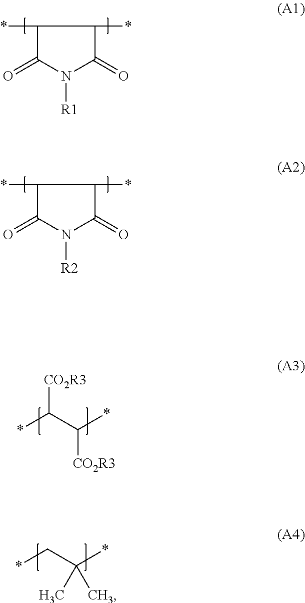

wherein
R1 represents an alkyl residue which may optionally be heterofunctionalized;
R2 represents an alkyl residue which differs from R1 and which may optionally be heterofunctionalized;
R3, independently of each other, represent alkyl residues which may optionally be heterofunctionalized and which differ from R1 and R2.
7. A cosmetic agent according to point 6, exemplified in that R1 in formula (A1) represents a residue —CH(CH₃)CH₂—(OCH(CH₃)CH₂)ₓ(O[CH₂]₂)_{y}OCH₃, in which x and y, independently of each other, have a value of between 1 and 100.
8. A cosmetic agent according to one of points 6 or 7, exemplified in that residue R2 in the structural unit (A2) represents a residue containing an amino group, preferably a residue with a tertiary amine.
9. A cosmetic agent according to one of points 6 to 8, exemplified in that at least one of the residues R3 of the structural unit (A3) represents an alkyl residue, preferably a (C1 to C4) alkyl residue.
10. A cosmetic agent according to one of the preceding points, exemplified in that the proportion by weight of the polyimide with respect to the total weight of the cosmetic agent is from about 0.5% to about 8.0% by weight, preferably from about 1.0% to about 6.0% by weight.
11. A cosmetic agent according to one of the preceding points, exemplified in that the agent further contains from about 0.1% to about 4.0% by weight, preferably from about 0.2% to about 3.0% by weight and in particular from about 0.5% to about 2.0% by weight of at least one surfactant.
12. A cosmetic agent according to one of the preceding points, exemplified in that the agent further contains at least one surfactant selected from the group of non-ionic, cationic and amphoteric surfactants.
13. A cosmetic agent according to one of the preceding points, exemplified in that the agent, with respect to its total weight, further contains from about 0.05% to about 2.0% by weight, preferably from about 0.1% to about 1.0% by weight and in particular from about 0.1% to about 0.5% by weight of at least one nonionic surfactant from the group formed by addition products of from about 30 to about 60 Mol ethylene oxide with castor oil and hydrogenated castor oil, preferably at least one nonionic surfactant from the group formed by compounds with the INCI names steareth-30, ceteareth-30, oleth-30, ceteareth-50, PEG-40 hydrogenated castor oil and PEG-60 hydrogenated castor oil, in particular PEG-40 hydrogenated castor oil.
14. A cosmetic agent according to one of the preceding points, exemplified in that the agent, with respect to its total weight, further contains from about 0.05% to about 2.0% by weight, advantageously from about 0.1% to about 1.0% by weight and in particular from about 0.1% to about 0.5% by weight of at least one cationic surfactant from the group of (C₁₂ to C₁₈)-alkyltrimethylammonium salt(s), preferably from the group of substances with the INCI name cetrimonium chloride.
15. A cosmetic agent according to one of the preceding points, exemplified in that the agent, with respect to its total weight, further contains from about 0.05% to about 2.0% by weight, advantageously from about 0.1% to about 1.0% by weight and in particular from about 0.1% to about 0.5% by weight of at least one amphoteric surfactant from the group of substances with the INCI name amidopropylbetaine, preferably from the group of substances with the INCI name cocoamidopropyl betaine.
16. A cosmetic agent according to one of the preceding points, exemplified in that the agent, with respect to its total weight, further contains from about 0.1% to about 5.0% by weight, preferably from about 0.2% to about 3.0% by weight and in particular from about 0.5% to about 2.0% by weight of at least one wax from the group carnauba wax (INCI: copernicia cerifera cera), myrica cerifera fruit wax (INCI), rhus verniciflua peel cera (INCI), beeswax (INCI: beeswax cera alba), lanolin (INCI: lanolin), microcrystalline wax and in particular mixtures thereof.
17. A cosmetic agent according to one of the preceding points, exemplified in that the agent, with respect to its total weight, further contains from about 0.1% to about 6.0% by weight, preferably from about 0.2% to about 4.0% by weight and in particular from about 0.5% to about 3.0% by weight of at least one non-ionic polymer which differs from the polyimide.
18. A cosmetic agent according to one of the preceding points, exemplified in that the agent, with respect to its total weight, further contains from about 0.1% to about 6.0% by weight, preferably from about 0.2% to about 4.0% by weight and in particular from about 0.5% to about 3.0% by weight of at least one non-ionic polymer from the group of vinylacetate/vinylpyrrolidone copolymers.
19. A cosmetic agent according to one of the preceding points, exemplified in that the agent, with respect to its total weight, further contains from about 0.1% to about 6.0% by weight, preferably from about 0.2% to about 4.0% by weight and in particular from about 0.5% to about 3.0% by weight of at least one non-ionic polymer from the group of vinylpyrrolidone homopolymers.
20. A cosmetic agent according to one of the preceding points, exemplified in that the agent, with respect to its total weight, further contains from about 0.1% to about 4.0% by weight, preferably from about 0.2% to about 3.0% by weight and in particular from about 0.3% to about 2.0% by weight of at least one polymeric thickening agent or polymeric gelling agent.
21. A cosmetic agent according to one of the preceding points, exemplified in that the cosmetic agent contains, with respect to its total weight, from about 50% to about 99% by weight, preferably from about 60% to about 98% by weight, particularly preferably from about 70% to about 97% by weight and in particular from about 80% to about 96% by weight of water or water-alcohol mixture.
22. A cosmetic agent according to one of the preceding points, exemplified in that the cosmetic agent, with respect to its total weight, contains from about 1% to about 15% by weight, particularly preferably from about 2% to about 12.5% by weight and in particular from about 3% to about 10% by weight of at least one propellant, preferably propane and/or butane.
23. Use of a cosmetic agent according to one of the preceding points, for the

temporary styling of keratinic fibers

straightening of keratinic fibers

improving the hold of a hairstyle

improving the manageability, in particular the combability.
24. A method for the temporary shaping of keratinic fibers, in particular human hair, in which a cosmetic agent according to one of the preceding points is applied to the keratinic fibers.
25. The method according to point 24, exemplified in that after application of the cosmetic agents, the human hair is temporarily shaped without carrying out a heat treatment using an external heat source, in particular a heat source with a temperature of above 150° C.

In a first aspect of this application, cosmetic agents are provided for the temporary styling of keratinic fibers, in particular human hair, containing, in a cosmetic support, with respect to its total weight,
a) from about 0.05% to about 5.0% by weight of at least one (meth)acrylamidopropyltrimethylammonium salt homo- or copolymer; and
b) from about 0.5% to about 10% by weight of polyimide.

The cosmetic agents as contemplated herein contain their substances in a cosmetic support, preferably in a cosmetic support that contains water, an alcoholic cosmetic support or a hydroalcoholic cosmetic support. For the purposes of temporary hair styling, examples of such supports are lotions, water-in-oil emulsions, oil-in-water emulsions, creams, gels, mousses, pomades, waxes or other preparations which are suitable for application to the hair.

As contemplated herein, it is preferable for the cosmetic support to be an aqueous cosmetic support or a hydroalcoholic cosmetic support. As contemplated herein, the cosmetic support of the agent as contemplated herein more preferably contains water so that the agent contains at least 50% by weight of water—with respect to the total weight of the agent.

The term “hydroalcoholic supports” as used in the context of the present disclosure should be understood to mean aqueous compositions which contain water and also C₁-C₄ alcohol, in particular ethanol or isopropanol, methoxybutanol, benzylalcohol, ethyldiglycol, 1,2-propyleneglycol or 1,3-propyleneglycol.

A preferred embodiment of the present disclosure is exemplified in that the cosmetic agents, with respect to their total weight, contain from about 50% to about 99% by weight, preferably from about 60 to about 98% by weight, particularly preferably from about 70% to about 97% by weight and in particular from about 80% to about 96% by weight of water or water-alcohol mixture.

A first essential component of the cosmetic agent as contemplated herein is the (meth)acrylamidopropyltrimethylammonium salt homo- or copolymer. Adding this cationic polymer to the cosmetic agents not only improves its care action, for example with respect to the combability of the hair, but also surprisingly improves the fixative effect of the agent; in other words, it reinforces the cosmetic action of the non-ionic, anionic or amphoteric film-forming polymers b).

(Meth)acrylamidopropyltrimethylammonium salt homopolymers as well as (meth)acrylamidopropyltrimethylammonium salt copolymers are equally applicable as the essential component a).

It has been shown to be cosmetically advantageous when a (3-acrylamidopropyl)trimethylammonium salt homopolymer, preferably a (3-acrylamidopropyl)trimethylammonium chloride homopolymer, is used as the (meth)acrylamidopropyltrimethylammonium salt homopolymer a). Cosmetic agents of this type are thus preferred in the present diclosure.

In a second group of preferred cosmetic agents, (3-methacrylamidopropyl)-trimethylammonium salt homopolymer is used as the (meth)acrylamidopropyltrimethylammonium salt homopolymer, preferably (3-methacrylamidopropyl)-trimethylammonium chloride homopolymer.

In a third group of preferred cosmetic agents, a (3-acrylamidopropyl)-trimethylammonium salt copolymer, preferably a (3-acrylamidopropyl)-trimethylammonium chloride-acrylamide copolymer, is used as the (meth)acrylamidopropyltrimethylammonium salt copolymer a).

Clearly, the cosmetic agents as contemplated herein may also contain mixtures of (3-acrylamidopropyl)trimethylammonium salt homopolymer and (3-methacrylamidopropyl)-trimethylammonium salt homopolymer, in particular mixtures of (3-acrylamidopropyl)trimethylammonium chloride homopolymer and (3-methacrylamidopropyl)-trimethylammonium chloride homopolymer.

Similarly, mixtures of (3-acrylamidopropyl)trimethylammonium salt copolymer and (3-methacrylamidopropyl)-trimethylammonium salt copolymer, in particular mixtures of (3-acrylamidopropyl)trimethylammonium chloride copolymer and (3-methacrylamidopropyl)-trimethylammonium chloride copolymer, may be used.

Because of its high positive charge density, the (meth)acrylamidopropyltrimethylammonium salt homo- or copolymer is capable of effectively binding to the negatively charged surface of human hair.

Preferred (meth)acrylamidopropyltrimethylammonium salt homopolymers have a mean molecular mass M[r] of approximately 300000.

Examples of suitable (meth)acrylamidopropyltrimethylammonium salt homopolymers are commercially available from Ashland under the trade name N-DurHance A-1000 (INCI: polyacrylamidopropyltrimonium chloride).

Particular (meth)acrylamidopropyltrimethylammonium salt copolymers are the copolymers of (meth)acrylamidopropyltrimethylammonium salts with non-ionic monomers. Particularly preferred non-ionic monomers are acrylamides.

As an example, suitable (meth)acrylamidopropyltrimethylammonium salt copolymers are freely commercially available from Ashland under the trade name N-Hance SP-100 (INCI: acrylamidopropyl trimonium chloride/acrylamide copolymer).

The proportion by weight of the (meth)acrylamidopropyltrimethylammonium salt homo- or copolymer, preferably of the (meth)acrylamidopropyltrimethylammonium salt homopolymer with respect to the total weight of the cosmetic agent is advantageously from about 0.05% to about 4.0% by weight, preferably from about 0.1% to about 3.0% by weight and in particular from about 0.1% to about 2.0% by weight.

The agents as contemplated herein contain a polyimide as the second essential component.

In a preferred embodiment, the polyimide contains at least one structural unit (Al), at least one structural unit (A2), at least one structural unit (A3) and at least one structural unit (A4),

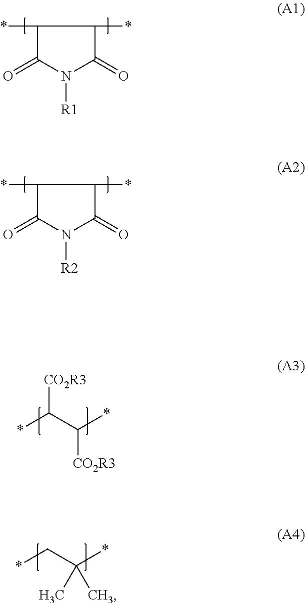

wherein
R1 represents an alkyl residue which may optionally be heterofunctionalized;
R2 represents an alkyl residue which differs from R1 and which may optionally be heterofunctionalized;
R3, independently of each other, represent alkyl residues which may optionally be heterofunctionalized and which differ from R1 and R2

Having regard to the cosmetic properties of the agents as contemplated herein, it has been shown to be advantageous when:

residue R1 in formula (A1) represents a residue —CH(CH₃)CH₂—(OCH(CH₃)CH₂)ₓ(O[CH₂]₂)₆OCH₃, in which x and y, independently of each other, have a value between 1 and about 100 and/or

residue R2 in the structural unit (A2) represents a residue containing an amino group, advantageously a residue with a tertiary amine, and/or

at least one of the residues R3 of the structural unit (A3) represents an alkyl residue, preferably a (C1 to C4) alkyl residue.

Particularly preferred polyimides contain at least 70% by weight, more preferably at least 80% by weight, preferably at least 90% by weight and in particular at least 95% by weight of structural units (A1), (A2), (A3) and (A4). More preferred copolymers A consist entirely of the structural units (A1), (A2), (A3) and (A4).

Some preferred polyimides are listed below. These polyimides contain at least 70% by weight, advantageously at least 80% by weight, preferably at least 90% by weight and in particular at least 95% by weight, and more particularly preferably consist entirely of the structural units (A1), (A2), (A3) and (A4):
A-I) Polyimides with at least one structural unit (A1), at least one structural unit (A2), at least one structural unit (A3) and at least one structural unit (A4),

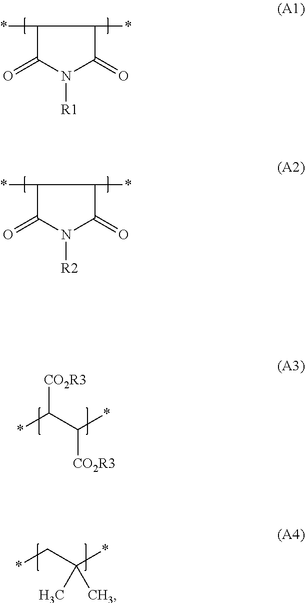

wherein
R1 in formula (A1) represents a residue —CH(CH₃)CH₂—(OCH(CH₃)CH₂)ₓ(O[CH₂]₂)_{y}OCH₃, in which x and y, independently of each other, have a value of between 1 and about 100;
R2 in formula (A2) represents a residue —(CH₂)₃—N(CH₃)₂;
R3, independently of each other, represent alkyl residues which may optionally be heterofunctionalized and which differ from R1 and R2.
A-II) Polyimides with at least one structural unit (A1), at least one structural unit (A2), at least one structural unit (A3) and at least one structural unit (A4),

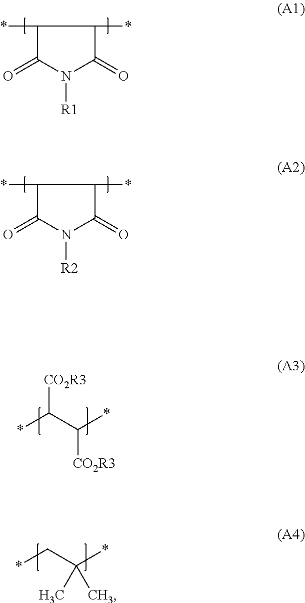

wherein
R1 in formula (A1) represents a residue —CH(CH₃)CH₂—(OCH(CH₃)CH₂)ₓ(O[CH₂]₂)_{y}OCH₃, in which x and y, independently of each other, have a value of between 1 and about 100;
R2 represents an alkyl residue which differs from R1 and which may optionally be heterofunctionalized;
one residue R3 in the formula (A3) represents —CH₂CH₃ or —CH₂CH₂CH₃, preferably —CH₂CH₃, and one residue R3 in formula (A3) represents H.
A-III) Polyimides with at least one structural unit (A1), at least one structural unit (A2), at least one structural unit (A3) and at least one structural unit (A4),

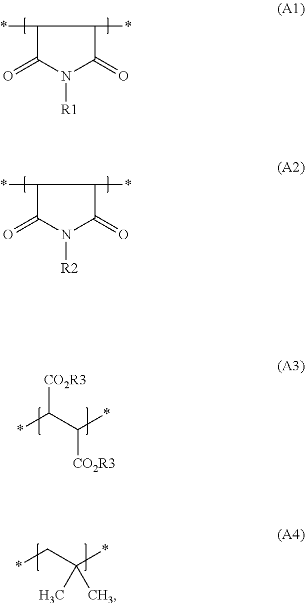

wherein
R1 represents an alkyl residue which may optionally be heterofunctionalized;
R2 in formula (A2) represents a residue —(CH₂)₃—N(CH₃)₂;
one residue R3 in the formula (A3) represents —CH2CH₃ or —CH₂CH₂CH₃, preferably —CH₂CH₃, and one residue R3 in formula (A3) represents H.

More particularly preferred polyimides are exemplified in that
the residue R1 in formula (A1) represents a residue —CH(CH₃)CH₂—(OCH(CH₃)CH₂)ₓ(O[CH₂]₂)_{y}OCH₃, in which x and y, independently of each other, have a value of between 1 and about 100;
the residue R2 in formula (A2) represents a residue —(CH₂)₃—N(CH₃)₂;
one residue R3 represents —CH₂CH₃ and one residue R3 in formula (A3) represents H.

Suitable polyimides are, for example, freely commercially available from ISP under the trade names Aquaflex XL 30 (INCI: polyimide-1).

The proportion by weight of the polyimide with respect to the total weight of the cosmetic agent is advantageously from about 0.5% to about 8.0% by weight and in particular from about 1.0% to about 6.0% by weight.

In addition to the two components described above, the cosmetic agent as contemplated herein may contain other substances or auxiliary agents, wherein particularly preferred substances or auxiliary agents are those which improve the manufacturability, applicability and/or cosmetic action of these agents.

In order to improve the manufacturability, applicability and/or cosmetic action, the cosmetic agents preferably contain surfactants, in particular non-ionic and/or cationic and/or amphoteric surfactants. The proportion by weight of surfactant with respect to the total weight of the cosmetic agent is advantageously from about 0.1% to about 4.0% by weight, preferably from about 0.2% to about 3.0% by weight and in particular from about 0.5% to about 2.0% by weight.

A first group of preferred surfactants are the non-ionic surfactants, wherein particularly preferred cosmetic agents are exemplified in that they contain, with respect to their total weight, from about 0.05% to about 2.0% by weight, advantageously from about 0.1% to about 1.0% by weight and in particular from about 0.1% to about 0.5% by weight of at least one nonionic surfactant, preferably at least one nonionic surfactant from the group formed by addition products of from about 30 to about 60 Mol ethylene oxide with castor oil and hydrogenated castor oil, advantageously at least one nonionic surfactant from the group formed by compounds with the INCI names steareth-30, ceteareth-30, oleth-30, ceteareth-50, PEG-40 hydrogenated castor oil and PEG-60 hydrogenated castor oil, in particular PEG-40 hydrogenated castor oil.

Preferred non-ionic surfactants are PEG derivatives of hydrogenated castor oil which, for example, are obtainable under the name PEG hydrogenated castor oil, for example PEG-30 hydrogenated castor oil, PEG-33 hydrogenated castor oil, PEG-35 hydrogenated castor oil, PEG-36 hydrogenated castor oil, PEG-40 hydrogenated castor oil or PEG-60 hydrogenated castor oil. Particularly preferred non-ionic surfactants as contemplated herein are those which are selected from the group of PEG derivatives of hydrogenated castor oil, particularly preferably from the group PEG-40 hydrogenated castor oil and PEG-60 hydrogenated castor oil, in particular PEG-40 hydrogenated castor oil.

Cationic surfactants form a further preferred component of the cosmetic agents as contemplated herein. Preferred cationic surfactants are selected from quaternary ammonium compounds, esterquats and amidoamines. The cationic surfactants are contained in the cosmetic agent, with respect to its total weight, in quantities of from about 0.05% to about 2.0% by weight, particularly preferably from about 0.1% to about 1.0% by weight and in particular from about 0.1% to about 0.5% by weight. Cationic surfactants from the quaternary ammonium compound group, preferably from the group of (C₁₂ to C₁₈)-alkyltrimethylammonium salt(s), in particular from the group of substances with the INCI name cetrimonium chloride, are preferred.

Preferred quaternary ammonium compounds are ammonium halides, in particular chlorides and bromides, such as alkyltrimethyl ammonium chloride, dialkyldimethylammonium chloride and trialkylmethylammonium chloride, for example cetyltrimethylammonium chloride, stearyltrimethylammonium chloride, distearyldimethylammonium chloride, lauryldimethylammonium chloride, lauryldimethylbenzylammonium chloride and tricetylmethylammonium chloride, as well as imidazolium compounds known by their INCI names quaternium-27 and quaternium-83. The long alkyl chains of the surfactants mentioned above preferably contain from about 10 to about 18 carbon atoms. More particularly preferred cosmetic agents as contemplated herein are exemplified in that they contain, with respect to their total weight, from about 0.05% to about 3.0% by weight, particularly preferably from about 0.1% to about 2.0% by weight and in particular from about 0.1% to about 1.0% by weight of (C₁₂ to C₁₈)-alkyltrimethylammonium salt(s).

The amphoteric surfactants used are those surface-active compounds which carry at least one quaternary ammonium group and at least one —COO⁽⁻⁾- or —SO₃⁽⁻⁾ group in their molecule. Particularly suitable zwitterionic surfactants and emulsification agents are what are known as betaines, such as N-alkyl-N,N-dimethylammonium glycinate, for example cocoalkyldimethylammonium glycinate, N-acylaminopropyl-N,N-dimethylammonium glycinate, for example cocoacylaminopropyldimethylammonium glycinate, and 2-alkyl-3-carboxymethyl-3-hydroxyethylimidazoline respectively containing 8 to about 18 carbon atoms in the alkyl or acyl group, as well as cocoacylaminoethylhydroxyethylcarboxymethyl glycinate. A preferred amphoteric surfactant is the fatty acid amide derivative known by the INCI name cocamidopropyl betaine.

Cosmetic agents which, with respect to their total weight, further contain from about 0.05% to about 2.0% by weight, advantageously from about 0.1% to about 1.0% by weight and in particular from about 0.1% to about 0.5% by weight of at least one amphoteric surfactant from the group of substances with the INCI name amidopropylbetaine, preferably from the group of substances with the INCI name cocoamidopropyl betaine, are distinguished by particularly advantageous cosmetic properties and are therefore preferred.

As a further optional component, preferred cosmetic agents contain at least one wax; moreover, waxes with a melting point of more than 40° C. are preferred.

The wax should be solid at 25° C. and should melt in a range >40° C. In preferred embodiments of the present disclosure, 50% by weight or more of the total quantity of wax (a), preferably from about 70% by weight to about 90% by weight of the total quantity of wax (a), has a melting point in the range from about 45° C. to about 60° C.

As contemplated herein, the natural or synthetic waxes used may be solid paraffins or isoparaffins, vegetable waxes such as candelila wax, carnauba wax, esparto grass wax, Japan wax, cork wax, cane sugar wax, ouricury wax, montan wax, sunflower wax, fruit waxes and animal waxes such as, for example, beeswax and other insect waxes, sperm whale oil, shellac wax, lanolin and rump fat, also mineral waxes such as, for example, ceresin and ozokerite or petrochemical waxes such as, for example petroleum jelly, paraffin waxes, microwaxes from polyethylene or polypropylene and polyethyleneglycol waxes. It may be advantageous to use hydrogenated waxes. Furthermore, chemically modified waxes may also be used, in particular hard waxes, for example montan ester waxes, Sasol waxes and hydrogenated jojoba waxes.

Cosmetic agents which, with respect to their total weight, further contain from about 0.1% to about 5.0% by weight, preferably from about 0.2% to about 3.0% by weight and in particular from about 0.5% to about 2.0% by weight of at least one wax from the group carnauba wax, (INCI: copernicia cerifera cera), myrica cerifera fruit wax (INCI), rhus verniciflua peel cera (INCI), beeswax (INCI: beeswax cera alba), lanolin (INCI: lanolin), microcrystalline wax and in particular mixtures thereof, are preferred because of their outstanding cosmetic action.

A further preferred component of the cosmetic agents as contemplated herein are film-forming polymers, in particular the non-ionic film-forming polymers. These film-forming polymers may be of synthetic or natural origin.

Preferred cosmetic agents contain, with respect to their total weight, from about 0.1% to about 6.0% by weight, preferably from about 0.2% to about 4.0% by weight and in particular from about 0.5% to about 3.0% by weight of non-ionic polymer. This non-ionic film-forming polymer differs from the polyimide.

A preferred group of nonionic film-forming polymers used in the cosmetic preparation are vinylpyrrolidone homo- or copolymers B. Examples of particularly preferred polymers are as follows:
polyvinylpyrrolidones, such as those marketed under the name Luviskol® (BASF),
vinylpyrrolidone/vinylester copolymers, such as those marketed under the trade name Luviskol® (BASF). Luviskol® VA 64 and Luviskol® VA 73, which are each vinylpyrrolidone/vinylacetate copolymers, are preferred non-ionic polymers.

Because of their outstanding cosmetic action in combination with (meth)acrylamidopropyltrimethylammonium salt homo- or copolymer and polyimide, polyvinylpyrrolidone (INCI name: PVP) as well as vinylpyrrolidone/vinylacetate copolymers (INCI name VP/VA copolymer) are particularly preferred.

In a first embodiment, these preferred cosmetic agents contain, with respect to their total weight, from about 0.1% to about 6.0% by weight, preferably from about 0.2% to about 4.0% by weight and in particular from about 0.5% to about 3.0% by weight of vinylpyrrolidone homopolymer.

In a second embodiment, these preferred cosmetic agents contain, with respect to their total weight, from about 0.1% to about 6.0% by weight, preferably from about 0.2% to about 4.0% by weight and in particular from about 0.5% to about 3.0% by weight of vinylacetate/vinylpyrrolidone copolymer.

In a third embodiment, these preferred cosmetic agents contain, with respect to their total weight, from about 0.1% to about 6.0% by weight, preferably from about 0.2% to about 4.0% by weight and in particular from about 0.5% to about 3.0% by weight of vinylpyrrolidone homopolymer as well as from about 0.1% to about 6.0% by weight, preferably from about 0.2% to about 4.0% by weight and in particular from about 0.5% to about 3.0% by weight of vinylacetate/vinylpyrrolidone copolymer.

In order to further improve the dispensing, application and use of the cosmetic agents as contemplated herein, they preferably contain a polymeric thickening agent or a polymeric gelling agent. The proportion by weight of the polymeric thickening agent or the polymeric gelling agent with respect to the total weight of the cosmetic agent is preferably from about 0.1% to about 4.0% by weight, preferably from about 0.2% to about 3.0% by weight and in particular from about 0.3% to about 2.0% by weight.

The thickening agent may, for example, be selected from the following known polymeric thickening agents listed with their INCI names: acrylamides copolymer, acrylamide/sodium crylate copolymer, acrylamide/sodium acryloyldimethyltaurate copolymer, acrylates/acetoacetoxyethyl methacrylate copolymer, acrylates/beheneth-25 methacrylate copolymer, acrylates/C 10-30 alkyl acrylate crosspolymer, acrylates/ceteth-20 itaconate copolymer, acrylates/ceteth-20 methacrylate copolymer, acrylates/laureth-25 methacrylate copolymer, acrylates/palmeth-25 acrylate copolymer, acrylates/palmeth-25 itaconate copolymer, acrylates/steareth-50 acrylate copolymer, acrylates/steareth-20 itaconate copolymer, acrylates/steareth-20 methacrylate copolymer, acrylates/stearyl methacrylate copolymer, acrylates/vinyl isodecanoate crosspolymer, acrylic acid/acrylonitrogens copolymer, agar, agarose, alcaligenes polysaccharides, algin, alginic acid, ammonium acrylates/acrylonitrogens copolymer, ammonium acrylates copolymer, ammonium acryloyldimethyltaurate/vinyl formamide copolymer, ammonium acryloyldimethyltaurate/VP copolymer, ammonium alginate, ammonium polyacryloyldimethyl taurate, amylopectin, ascorbyl methylsilanol pectinate, astragalus gummifer gum, attapulgite, avena sativa (oat) kernel flour, bentonite, butoxy chitosan, caesalpinia spinosa gum, calcium alginate, calcium carboxymethyl cellulose, calcium carrageenan, calcium potassium carbomer, calcium starch octenylsuccinate, C20-40 alkyl stearate, carbomer, carboxybutyl chitosan, carboxymethyl chitin, carboxymethyl chitosan, carboxymethyl dextran, carboxymethyl hydroxyethylcellulose, carboxymethyl hydroxypropyl guar, cellulose acetate propionate carboxylate, cellulose gum, ceratonia siliqua gum, cetyl hydroxyethylcellulose, cholesterol/HDI/pullulan copolymer, cholesteryl hexyl dicarbamate pullulan, cyamopsis tetragonoloba (guar) gum, diglycol/CHDM/isophthalates/SIP copolymer, dihydrogenated tallow benzylmonium hectorite, dimethicone crosspolymer-2, dimethicone propyl PG-betaine, DMAPA acrylates/acrylic acid/acrylonitrogens copolymer, ethylene/sodium acrylate copolymer, gelatin, gellan gum, glyceryl alginate, glycine soja (Soybean) flour, guar hydroxypropyltrimonium chloride, hectorite, hydrated silica, hydrogenated potato starch, hydroxybutyl methylcellulose, hydroxyethyl acrylate/sodium acryloyldimethyl taurate copolymer, hydroxyethylcellulose, hydroxyethyl chitosan, hydroxyethyl ethylcellulose, hydroxypropylcellulose, hydroxypropyl chitosan, hydroxypropyl ethylenediamine carbomer, hydroxypropyl guar, hydroxypropyl methylcellulose, hydroxypropyl methylcellulose stearoxy ether, hydroxystearamide MEA, isobutylene/sodium maleate copolymer, methoxy PEG-22/dodecyl glycol copolymer, methylcellulose, methyl ethylcellulose, methyl hydroxyethylcellulose, microcrystalline cellulose, montmorillonite, natto gum, nonoxynyl hydroxyethylcellulose, octadecene/MA copolymer, pectin, PEG-800, PEG-crosspolymer, PEG-150/decyl alcohol/SMDI copolymer, PEG-175 diisostearate, PEG-190 distearate, PEG-15 glyceryl tristearate, PEG-140 glyceryl tristearate, PEG-240/HDI copolymer bis-decyltetradeceth-20 ether, PEG-100/IPDI copolymer, PEG-180/1aureth-50/TMMG copolymer, PEG-10/lauryl dimethicone crosspolymer, PEG-15/lauryl dimethicone crosspolymer, PEG-2M, PEG-5M, PEG-7M, PEG-9M, PEG-14M, PEG- 20M, PEG-23M, PEG-25M, PEG-45M, PEG-65M, PEG-90M, PEG-1 15M, PEG-160M, PEG-120 methyl glucose trioleate, PEG-180/octoxynol-40/TMMG copolymer, PEG-150 pentaerythrityl tetrastearate, PEG-4 rapeseedamide, PEG-150/stearyl alcohol/SMDI copolymer, polyacrylate-3, polyacrylic acid, polycyclopentadiene, polyether-1, polyethylene/isopropyl maleate/MA copolyol, polymethacrylic acid, polyquaternium-52, polyvinyl alcohol, potassium alginate, potassium aluminum polyacrylate, potassium carbomer, potassium carrageenan, potassium polyacrylate, potato starch modified, PPG-14 laureth-60 hexyl dicarbamate, PPG-14 laureth-60 isophoryl dicarbamate, PPG-14 palmeth-60 hexyl dicarbamate, propylene glycol alginate, PVP/decene copolymer, PVP montmorillonite, rhizobian gum, ricinoleic acid/adipic acid/AEEA copolymer, sclerotium gum, sodium acrylate/acryloyldimethyl taurate copolymer, sodium acrylates/acrolein copolymer, sodium acrylates/acrylonitrogens copolymer, sodium acrylates copolymer, sodium acrylates/vinyl isodecanoate crosspolymer, sodium acrylate/vinyl alcohol copolymer, sodium carbomer, sodium carboxymethyl chitin, sodium carboxymethyl dextran, sodium carboxymethyl beta-glucan, sodium carboxymethyl starch, sodium carrageenan, sodium cellulose sulfate, sodium cyclodextrin sulfate, sodium hydroxypropyl starch phosphate, sodium isooctylene/MA copolymer, sodium magnesium fluorosilicate, sodium polyacrylate, sodium polyacrylate starch, sodium polyacryloyldimethyl taurate, sodium polymethacrylate, sodium polystyrene sulfonate, sodium silicoaluminate, sodium starch octenylsuccinate, sodium stearoxy PG-hydroxyethylcellulose sulfonate, sodium styrene/acrylates copolymer, sodium tauride acrylates/acrylic acid/acrylonitrogens copolymer, solanum tuberosum (potato) starch, starch/acrylates/acrylamide copolymer, starch hydroxypropyltrimonium chloride, steareth-60 cetyl ether, steareth-100/PEG-136/HDI copolymer, sterculia urens gum, tamarindus indicaseed gum, tapioca starch, TEA-alginate, TEA-carbomer, triticum vulgare (wheat) starch, tromethamine acrylates/acrylonitrogens copolymer, welan gum, yeast beta-glucan, yeast polysaccharides, zea mays (corn) starch.

Particularly preferably, the thickening agent or the gelling agent is selected from polyacrylic acids (INCI: carbomer).

The agent may, for example, contain at least one protein hydrolysate and/or a derivative thereof as the care product. Protein hydrolysates are product mixtures which are obtained by the acidic, basic or enzymatically catalysed decomposition of proteins. The term “protein hydrolysate” as used in the present disclosure should also be understood to include total hydrolysates as well as individual amino acids and their derivatives, as well as mixtures of the various amino acids. The molecular weight of the protein hydrolysates used as contemplated herein is between about 75, the molecular weight of glycine, and about 200000; preferably, the molecular weight is from about 75 to about 50000, and more particularly preferably from about 75 to about 20000 Dalton.

The agent as contemplated herein may also contain at least one vitamin, a provitamin, a vitamin precursor and/or one of its derivatives as the care product. In this regard, as contemplated herein, those vitamins, provitamins and vitamin precursors are preferred which usually are categorized into the groups A, B, C, E, F and H.

Other care products are panthenol, caffeine, nicotinamide and sorbitol.

The agent as contemplated herein may also contain at least one plant extract as the care products, as well as mono- or oligosaccharides and/or lipids.

The composition of a number of particularly preferred cosmetic agents can be obtained from the following tables (information as the % by weight with respect to the total weight of the cosmetic agent, unless indicated otherwise).

| | Formula 1 | Formula 2 | Formula 3 | Formula 4 |
|---|---|---|---|---|
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Water | 50 to 99 | 60 to 98 | 70 to 97 | 80 to 96 |
| Other ingredients | qs 100 | qs 100 | qs 100 | qs 100 |
| | Formula 6 | Formula 7 | Formula 8 | Formula 9 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Water | 50 to 99 | 60 to 98 | 70 to 97 | 80 to 96 |
| Other ingredients | qs 100 | qs 100 | qs 100 | qs 100 |
| | Formula | Formula | Formula | Formula |
| | 11 | 12 | 13 | 14 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide-1 | 0.5 to 10 | 0.5 to 8.0 | 0.7 to 7.0 | 1.0 to 6.0 |
| Water | 50 to 99 | 60 to 98 | 70 to 97 | 80 to 96 |
| Other ingredients | qs 100 | qs 100 | qs 100 | qs 100 |
| | | Formula | | Formula |
| | Formula 16 | 17 | Formula 18 | 19 |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Water | 50 to 99 | 60 to 98 | 70 to 97 | 80 to 96 |
| Other ingredients | qs 100 | qs 100 | qs 100 | qs 100 |
| | | Formula | | Formula |
| | Formula 21 | 22 | Formula 23 | 24 |
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Water | 50 to 99 | 60 to 98 | 70 to 97 | 80 to 96 |
| Other ingredients | qs 100 | qs 100 | qs 100 | qs 100 |
| | Formula | Formula | Formula | Formula |
| | 26 | 27 | 28 | 29 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Water | 50 to 99 | 60 to 98 | 70 to 97 | 80 to 96 |
| Other ingredients | qs 100 | qs 100 | qs 100 | qs 100 |
| | Formula | Formula | Formula | Formula |
| | 31 | 32 | 33 | 34 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide-1 | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Water | 50 to 99 | 60 to 98 | 70 to 97 | 80 to 96 |
| Other ingredients | qs 100 | qs 100 | qs 100 | qs 100 |
| | | Formula | | Formula |
| | Formula 36 | 37 | Formula 38 | 39 |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Water | 50 to 99 | 60 to 98 | 70 to 97 | 80 to 96 |
| Other ingredients | qs 100 | qs 100 | qs 100 | qs 100 |
| | | Formula | | Formula |
| | Formula 41 | 42 | Formula 43 | 44 |
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Nonionic, film-forming polymer | 0.1 to 6.0 | 0.2 to 4.0 | 0.2 to 4.0 | 0.5 to 3.0 |
| Water | 50 to 99 | 60 to 98 | 70 to 97 | 80 to 96 |
| Other ingredients | qs 100 | qs 100 | qs 100 | qs 100 |
| | Formula | Formula | Formula | Formula |
| | 46 | 47 | 48 | 49 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Nonionic, film-forming polymer | 0.1 to 6.0 | 0.2 to 4.0 | 0.2 to 4.0 | 0.5 to 3.0 |
| Water | 50 to 99 | 60 to 98 | 70 to 97 | 80 to 96 |
| Other ingredients | qs 100 | qs 100 | qs 100 | qs 100 |
| | Formula | Formula | Formula | Formula |
| | 51 | 52 | 53 | 54 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide-1 | 0.5 to 10 | 0.5 to 8.0 | 0.7 to 7.0 | 1.0 to 6.0 |
| Nonionic, film-forming polymer | 0.1 to 6.0 | 0.2 to 4.0 | 0.2 to 4.0 | 0.5 to 3.0 |
| Water | 50 to 99 | 60 to 98 | 70 to 97 | 80 to 96 |
| Other ingredients | qs 100 | qs 100 | qs 100 | qs 100 |
| | | Formula | | Formula |
| | Formula 56 | 57 | Formula 58 | 59 |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Nonionic, film-forming polymer | 0.1 to 6.0 | 0.2 to 4.0 | 0.2 to 4.0 | 0.5 to 3.0 |
| Water | 50 to 99 | 60 to 98 | 70 to 97 | 80 to 96 |
| Other ingredients | qs 100 | qs 100 | qs 100 | qs 100 |
| | | Formula | | Formula |
| | Formula 61 | 62 | Formula 63 | 64 |
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Polymeric thickening agent or | 0.1 to 4.0 | 0.2 to 3.0 | 0.2 to 3.0 | 0.3 to 2.0 |
| polymeric gelling agent | | | | |
| Water | 50 to 99 | 60 to 98 | 70 to 97 | 80 to 96 |
| Other ingredients | qs 100 | qs 100 | qs 100 | qs 100 |
| | Formula | Formula | Formula | Formula |
| | 66 | 67 | 68 | 69 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Polymeric thickening agent or | 0.1 to 4.0 | 0.2 to 3.0 | 0.2 to 3.0 | 0.3 to 2.0 |
| polymeric gelling agent | | | | |
| Water | 50 to 99 | 60 to 98 | 70 to 97 | 80 to 96 |
| Other ingredients | qs 100 | qs 100 | qs 100 | qs 100 |
| | Formula | Formula | Formula | Formula |
| | 71 | 72 | 73 | 74 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide-1 | 0.5 to 10 | 0.5 to 8.0 | 0.7 to 7.0 | 1.0 to 6.0 |
| Polymeric thickening agent or | 0.1 to 4.0 | 0.2 to 3.0 | 0.2 to 3.0 | 0.3 to 2.0 |
| polymeric gelling agent | | | | |
| Water | 50 to 99 | 60 to 98 | 70 to 97 | 80 to 96 |
| Other ingredients | qs 100 | qs 100 | qs 100 | qs 100 |
| | | Formula | | Formula |
| | Formula 76 | 77 | Formula 78 | 79 |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Polymeric thickening agent or | 0.1 to 4.0 | 0.2 to 3.0 | 0.2 to 3.0 | 0.3 to 2.0 |
| polymeric gelling agent | | | | |
| Water | 50 to 99 | 60 to 98 | 70 to 97 | 80 to 96 |
| Other ingredients | qs 100 | qs 100 | qs 100 | qs 100 |
| | | Formula | | Formula |
| | Formula 81 | 82 | Formula 83 | 84 |
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Nonionic, film-forming polymer | 0.1 to 6.0 | 0.2 to 4.0 | 0.2 to 4.0 | 0.5 to 3.0 |
| Water | 50 to 99 | 60 to 98 | 70 to 97 | 80 to 96 |
| Other ingredients | qs 100 | qs 100 | qs 100 | qs 100 |
| | Formula | Formula | Formula | Formula |
| | 86 | 87 | 88 | 89 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Nonionic, film-forming polymer | 0.1 to 6.0 | 0.2 to 4.0 | 0.2 to 4.0 | 0.5 to 3.0 |
| Water | 50 to 99 | 60 to 98 | 70 to 97 | 80 to 96 |
| Other ingredients | qs 100 | qs 100 | qs 100 | qs 100 |
| | Formula | Formula | Formula | Formula |
| | 91 | 92 | 93 | 94 |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide-1 | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Nonionic, film-forming polymer | 0.1 to 6.0 | 0.2 to 4.0 | 0.2 to 4.0 | 0.5 to 3.0 |
| Water | 50 to 99 | 60 to 98 | 70 to 97 | 80 to 96 |
| Other ingredients | qs 100 | qs 100 | qs 100 | qs 100 |
| | | Formula | | Formula |
| | Formula 96 | 97 | Formula 98 | 99 |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Nonionic, film-forming polymer | 0.1 to 6.0 | 0.2 to 4.0 | 0.2 to 4.0 | 0.5 to 3.0 |
| Water | 50 to 99 | 60 to 98 | 70 to 97 | 80 to 96 |
| Other ingredients | qs 100 | qs 100 | qs 100 | qs 100 |

The cosmetic agents as contemplated herein are preferably packaged in a dispensing device which constitutes either a compressed gas container additionally filled with a propellant (aerosol container) or a non-aerosol container. The compressed gas container, with the aid of which a product is distributed by employing the internal gas pressure of the container via a valve, is defined as an “aerosol container”. In contrast to this definition, a “non-aerosol container” is a container which is under normal pressure, with the aid of which product is distributed by mechanical action using a pump or squeezing system.

In this regard, the agent as contemplated herein preferably additionally contains at least one propellant. Agents as contemplated herein which are in the form of an aerosol product can be manufactured in the normal manner. As a rule, all of the components of the agent as contemplated herein with the exception of the propellant are placed in a suitable pressure-tight container. This is then sealed with a valve. Finally, the desired quantity of propellant is added using the usual techniques.

In the embodiment as an aerosol mousse, the propellants suitable for use in the present disclosure are, for example, selected from N₂O, dimethylether, CO₂, air, alkanes containing from 3 to 5 carbon atoms such as propane, n-butane, iso-butane, n-pentane and iso-pentane, and mixtures thereof.

In accordance with the embodiment in the form of an aerosol mousse, the cited alkanes, mixtures of the cited alkanes or mixtures of the cited alkanes with dimethylether are used as the only propellant. However, the present disclosure also expressly encompasses the simultaneous use of propellants of the chlorofluorocarbon nature, in particular fluorocarbons. Dimethylether, propane, n-butane, iso-butane and mixtures thereof are preferred. More particularly preferably, mixtures of propane and butane are used as the only propellant in proportions by weight of propane to butane of from about 70:30 to about 15:85. As contemplated herein, the term “butane” should be understood to mean n-butane, iso-butane and mixtures of n-butane and iso-butane.

The propellants in the cosmetic agents as contemplated herein are preferably used in a quantity of from about 1% to about 15% by weight, particularly preferably from about 2% to about 12.5% by weight and in particular from about 3% to about 10% by weight—with respect to the total weight of the agent—wherein propane, butane and propane/butane mixtures are preferred.

The compositions of more preferred cosmetic compositions can be obtained from the following tables (information as the % by weight with respect to the total weight of the composition, unless indicated otherwise).

| | | Formula | | Formula |
|---|---|---|---|---|
| | Formula 1a | 2a | Formula 3a | 4a |
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 6a | 7a | 8a | 9a |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 11a | 12a | 13a | 14a |
| Acrylamidopropyltrimethylammonium | 0.05 to 5.0 | 0.05 to 4.0 | 0.1 to 3.0 | 0.1 to 2.0 |
| salt homopolymer | | | | |
| polyimides-1 | 0.5 to 10 | 0.5 to 8.0 | 0.7 to 7.0 | 1.0 to 6.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 16a | 17a | 18a | 19a |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 21a | 22a | 23a | 24a |
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 26a | 27a | 28a | 29a |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Water | 50 to 98 | 60 to 97 | 70 to 96 | 80 to 95 |
| Propellant, in particular | 1.0 to 15 | 2.0 to | 2.0 to 12.5 | 3.0 to 10 |
| propane/butane | | 12.5 | | |
| Other ingredients | qs 100 | qs 100 | qs 100 | qs 100 |
| | Formula | Formula | Formula | Formula |
| | 31a | 32a | 33a | 34a |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide-1 | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 36a | 37a | 38a | 39a |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 41a | 42a | 43a | 44a |
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Nonionic, film-forming polymer | 0.1 to 6.0 | 0.2 to 4.0 | 0.2 to 4.0 | 0.5 to 3.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 46a | 47a | 48a | 49a |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Nonionic, film-forming polymer | 0.1 to 6.0 | 0.2 to 4.0 | 0.2 to 4.0 | 0.5 to 3.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 51a | 52a | 53a | 54a |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide-1 | 0.5 to 10 | 0.5 to 8.0 | 0.7 to 7.0 | 1.0 to 6.0 |
| Nonionic, film-forming polymer | 0.1 to 6.0 | 0.2 to 4.0 | 0.2 to 4.0 | 0.5 to 3.0 |
| Water | 50 to 98 | 60 to 97 | 70 to 96 | 80 to 95 |
| Propellant, in particular | 1.0 to 15 | 2.0 to | 2.0 to 12.5 | 3.0 to 10 |
| propane/butane | | 12.5 | | |
| Other ingredients | qs 100 | qs 100 | qs 100 | qs 100 |
| | Formula | Formula | Formula | Formula |
| | 56a | 57a | 58a | 59a |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Nonionic, film-forming polymer | 0.1 to 6.0 | 0.2 to 4.0 | 0.2 to 4.0 | 0.5 to 3.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 61a | 62a | 63a | 64a |
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Polymeric thickening agent or | 0.1 to 4.0 | 0.2 to 3.0 | 0.2 to 3.0 | 0.3 to 2.0 |
| polymeric gelling agent | | | | |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 66a | 67a | 68a | 69a |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Polymeric thickening agent or | 0.1 to 4.0 | 0.2 to 3.0 | 0.2 to 3.0 | 0.3 to 2.0 |
| polymeric gelling agent | | | | |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 71a | 72a | 73a | 74a |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide-1 | 0.5 to 10 | 0.5 to 8.0 | 0.7 to 7.0 | 1.0 to 6.0 |
| Polymeric thickening agent or | 0.1 to 4.0 | 0.2 to 3.0 | 0.2 to 3.0 | 0.3 to 2.0 |
| polymeric gelling agent | | | | |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 76a | 77a | 78a | 79a |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Polymeric thickening agent or | 0.1 to 4.0 | 0.2 to 3.0 | 0.2 to 3.0 | 0.3 to 2.0 |
| polymeric gelling agent | | | | |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 81a | 82a | 83a | 84a |
| (Meth)acrylamidopropyltrimethyl- | 0.05 to 5.0 | 0.05 to 4.0 | 0.1 to 3.0 | 0.1 to 2.0 |
| ammonium salt homo- or | | | | |
| copolymer | | | | |
| Polyimide | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Nonionic, film-forming polymer | 0.1 to 6.0 | 0.2 to 4.0 | 0.2 to 4.0 | 0.5 to 3.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 86a | 87a | 88a | 89a |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Nonionic, film-forming polymer | 0.1 to 6.0 | 0.2 to 4.0 | 0.2 to 4.0 | 0.5 to 3.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 91a | 92a | 93a | 94a |
| Acrylamidopropyltrimethylammonium | 0.05 to 5.0 | 0.05 to 4.0 | 0.1 to 3.0 | 0.1 to 2.0 |
| salt homopolymer | | | | |
| Polyimide-1 | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Nonionic, film-forming polymer | 0.1 to 6.0 | 0.2 to 4.0 | 0.2 to 4.0 | 0.5 to 3.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |
| | Formula | Formula | Formula | Formula |
| | 96a | 97a | 98a | 99a |
| Polyacrylamidopropyltrimonium | 0.05 to 5.0 | 0.05 to 4.0 | 0.1 to 3.0 | 0.1 to 2.0 |
| chloride | | | | |
| Polyimide-1 | 0.5 to 10 | 0.5 to 8.0 | 0.7 to 7.0 | 1.0 to 6.0 |
| Nonionic surfactant | 0.05 to 2.0 | 0.1 to 2.0 | 0.1 to 1.0 | 0.1 to 0.5 |
| Nonionic, film-forming polymer | 0.1 to 6.0 | 0.2 to 4.0 | 0.2 to 4.0 | 0.5 to 3.0 |
| Water | 50 to 98 | 60 to 97 | 70 to 96 | 80 to 95 |
| Propellant, in particular | 1.0 to 15 | 2.0 to 12.5 | 2.0 to 12.5 | 3.0 to 10 |
| propane/butane | | | | |
| Other ingredients | qs 100 | qs 100 | qs 100 | qs 100 |

As already mentioned, the cosmetic compositions as contemplated herein are of particular application for the

temporary styling of keratinic fibers

straightening of keratinic fibers

improving the hold of a hairstyle

improving the manageability, in particular the combability.

The corresponding uses of the agents as contemplated herein therefore constitute further objectives of this application.

A final objective of the application is constituted by a method for the temporary shaping of keratinic fibers, in particular human hair, in which a cosmetic agent as contemplated herein is applied to the keratinic fibers. In a preferred embodiment of this method, after application of the cosmetic agent, the human hair is temporarily shaped without having to apply to the hair a heat treatment using an external heat source, in particular a heat source with a temperature of more than 150° C.

While at least one exemplary embodiment has been presented in the foregoing detailed description, it should be appreciated that a vast number of variations exist. It should also be appreciated that the exemplary embodiment or exemplary embodiments are only examples, and are not intended to limit the scope, applicability, or configuration of the various embodiments in any way. Rather, the foregoing detailed description will provide those skilled in the art with a convenient road map for implementing an exemplary embodiment as contemplated herein. It being understood that various changes may be made in the function and arrangement of elements described in an exemplary embodiment without departing from the scope of the various embodiments as set forth in the appended claims.

1. A cosmetic agent for the temporary styling of keratinic fibres, in particular human hair, comprising, in a cosmetic support, with respect to its total weight,
a) from about 0.05% to about 5.0% by weight of at least one (meth)acrylamidopropyltrimethylammonium salt homo- or copolymer; and
b) from about 0.5% to about 10% by weight of polyimide.

2. The cosmetic agent according to claim 1, wherein the cosmetic agent comprises a (meth)acrylamidopropyltrimethylammonium salt homopolymer as the (meth)acrylamidopropyltrimethylammonium salt homo- or copolymer a).

3. The cosmetic agent according to claim 1, wherein the proportion by weight of the (meth)acrylamidopropyltrimethylammonium salt homo- or copolymer a), with respect to the total weight of the cosmetic agent is from about 0.05% to about 4.0% by weight.

4. A The cosmetic agent according to claim 1, wherein the polyimide comprises at least one structural unit (A1), at least one structural unit (A2), at least one structural unit (A3) and at least one structural unit (A4), 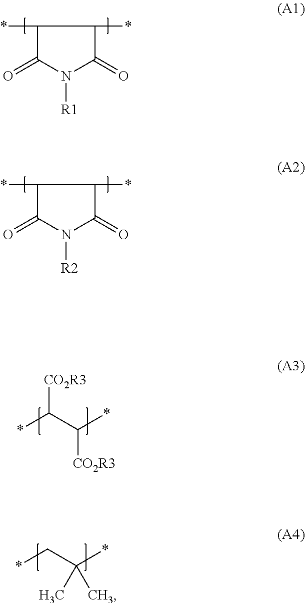
wherein
R1 represents an alkyl residue which may optionally be heterofunctionalized;
R2 represents an alkyl residue which differs from R1 and which may optionally be heterofunctionalized;
R3, independently of each other, represent alkyl residues which may optionally be heterofunctionalized and which differ from R1 and R2.

5. The cosmetic agent according to claim 1, wherein the proportion by weight of the polyimide with respect to the total weight of the cosmetic agent is from about 0.5% to about 8.0% by weight.

6. The cosmetic agent according to claim 1, wherein the agent, with respect to its total weight, further comprises from about 0.05% to about 2.0% by weight of at least one cationic surfactant selected from the group of (C₁₂ to C₁₈)-alkyltrimethylammonium salt(s).

7. The cosmetic agent according to claim 1, wherein the cosmetic agent, with respect to its total weight, comprises from about 50% to about 99% by weight of water or water-alcohol mixture.

8. A The cosmetic agent according to claim 1, wherein the cosmetic agent, with respect to its total weight, comprises from about 1% to about 15% by weight of at least one propellant.

9. The cosmetic agent according to claim 1, wherein the cosmetic agent is used for the purposes of:
temporary styling of keratinic fibres
straightening of keratinic fibres
improving the hold of a hairstyle and
improving the manageability, wherein manageability comprises combability.

10. A method for the temporary shaping of keratinic fibres, in particular human hair, in which a cosmetic agent is applied to the keratinic fibres; wherein the cosmetic agent comprises, in a cosmetic support, with respect to its total weight,
a) from about 0.05% to about 5.0% by weight of at least one (meth)acrylamidopropyltrimethylammonium salt homo- or copolymer; and
b) from about 0.5% to about 10% by weight of polyimide.

11. The cosmetic agent according to claim 1, wherein the (meth)acrylamidopropyltrimethylammonium salt homo- or copolymer a) comprises (3-acrylamidopropyl)-trimethylammonium salt homopolymer

12. The cosmetic agent according to claim 1, wherein the (meth)acrylamidopropyltrimethylammonium salt homo- or copolymer a) comprises (3-acrylamidopropyl)-trimethylammonium chloride homopolymer.

13. The cosmetic agent according to claim 1, wherein the proportion by weight of the (meth)acrylamidopropyltrimethylammonium salt homo- or copolymer a), with respect to the total weight of the cosmetic agent, is from about 0.05% to about 4.0% by weight.

14. The cosmetic agent according to claim 1, wherein the proportion by weight of the (meth)acrylamidopropyltrimethylammonium salt homo- or copolymer a), with respect to the total weight of the cosmetic agent, is from about 0.1% to about 3.0% by weight.

15. The cosmetic agent according to claim 1, wherein the proportion by weight of the (meth)acrylamidopropyltrimethylammonium salt homo- or copolymer a), with respect to the total weight of the cosmetic agent, is from about 0.1% to about 2.0% by weight.

16. The cosmetic agent according to claim 1, wherein the proportion by weight of the polyimide with respect to the total weight of the cosmetic agent is from about 1.0% to about 6.0% by weight.

17. The cosmetic agent according to claim 6, wherein the cationic surfactant is selected from the group of substances with the INCI name cetrimonium chloride.

18. The cosmetic agent according to claim 1, wherein the cosmetic agent, with respect to the total weight of the cosmetic agent, comprises at from about 80% to about 97% by weight of a water or water-alcohol mixture.

19. The cosmetic agent according to claim 1, wherein the cosmetic agent, with respect to the total weight of the cosmetic agent, comprises from about 3% to about 10% by weight of at least one propellant.

20. The cosmetic agent according to claim 19, wherein the propellant comprises propane and/or butane.
